(19) United States
(12) Patent Application Publication
(10) Pub. No.: US 2009/0000614 A1
(43) Pub. Date: Jan. 1, 2009

(54) Inflatable pad for medical system

(76) Inventor: Aaron W. Carrano, San Jose, CA (US)

Correspondence Address: BLAKELY, SOKOLOFF, TAYLOR & ZAFMAN LLP, Seventh Floor, 12400 Wilshire Boulevard, Los Angeles, CA, 90025, US

(21) Appl. No.: 11/824,103

(22) Filed: Jun. 29, 2007

(51) Int. Cl. A61F 5/34

(52) U.S. Cl. 128/118.1

(57) ABSTRACT

Systems and methods for positioning a patient on a treatment couch of a surgical system are disclosed. The system includes a pressure sensor and a pad having a plurality of selectively inflatable chambers. The inflation of the chambers can be dynamically and/or automatically adjusted in response to sensed pressure changes.

### TECHNICAL FIELD

This invention relates to the field of medical systems and, in particular, to an inflatable pad for a medical system.

### BACKGROUND

Many medical procedures require patients to sit or lie for relatively large periods of time. These medical procedures sometimes require that patients be immobile as well. Laying on a flat, hard table as required for these procedures, for an extended amount of time can cause discomfort for the patients. Similarly, remaining immobile for extended periods of time can be difficult and uncomfortable for many patients.

Some medical systems provide a foam pad to improve comfort for the patient. The foam pad, however, is not customizable to individual patients, nor does it immobilize patients, nor is it adjustable. To immobilize patients, sealable plastic bags are sometimes employed. A vacuum removes air from the bag to restrict patient movement. These plastic bags, however, provide no comfort to the patient.

### BRIEF DESCRIPTION OF THE DRAWINGS

Embodiments of the invention are described by way of example with reference to the accompanying drawings, wherein:

FIG. 1 is a perspective view of a treatment delivery system in accordance with one embodiment of the invention;

FIG. 2 is a block diagram showing the relationship of components of a treatment delivery system in accordance with one embodiment of the invention;

FIG. 3 is a side view of a treatment couch and pad in accordance with one embodiment of the invention;

FIG. 4 is a detailed view of an exemplary pressure sensor in accordance with one embodiment of the invention;

FIGS. 5A-5D are top views of the pad illustrating exemplary chamber layouts in accordance with embodiments of the invention;

FIGS. 6A-6B are schematic views of the pad illustrating exemplary mechanical arrangements in accordance with embodiments of the invention;

FIG. 7A is a block diagram of a system for the pad in accordance with one embodiment of the invention;

FIG. 7B is a block diagram of a patient positioning system in accordance with one embodiment of the invention;

FIG. 8 is a schematic view of an exemplary user interface in accordance with one embodiment of the invention; and

FIG. 9 is a flow diagram illustrating a process for using the pad system in accordance with one embodiment of the invention.

### DETAILED DESCRIPTION

Embodiments of systems, devices and methods for treatment of a patient are described herein. In the following description numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places through this specification are not necessarily all referring to the same embodiment. Furthermore, the particular features, structures, or characteristics may be combined in any suitable manner in one or more embodiments.

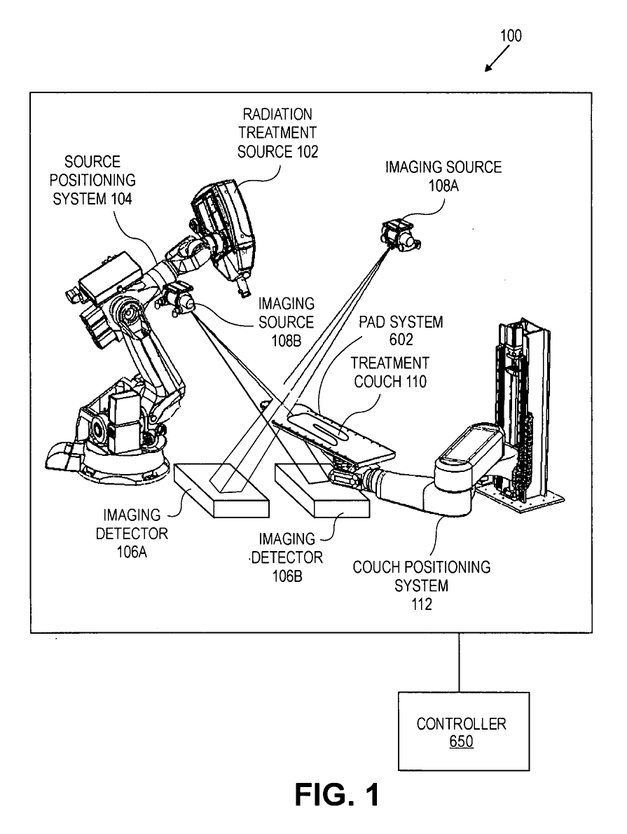

FIG. 1 is a perspective view of an image guided radiation treatment delivery system 100, in accordance with one embodiment of the invention. The illustrated embodiment of the radiation treatment delivery system 100 includes a radiation treatment source 102, a source positioning system 104, imaging detectors 106A and 106B (collectively 106, also referred to as imagers), imaging sources 108A and 108B, a treatment couch 110 and a couch positioning system 112.

System 100 may be used to perform radiotherapy or radiosurgery to treat or ablate lesions within a patient. During radiation treatment, the patient rests on treatment couch 110, which is maneuvered to position a volume of interest (“VOI”) within a patient to a preset position or within an operating range accessible to radiation treatment source 102 (e.g., field of view). Similarly, radiation treatment source 102 is maneuvered with multiple degrees of freedom (e.g., rotational and translation freedom) to one or more locations during delivery of a treatment plan. At each location, radiation treatment source 102 may deliver a dose of radiation as prescribed by a treatment plan.

Imaging sources 108 and imaging detectors 106 are part of an image guidance system that provides input to a control system that control the position of treatment couch 110 and/or radiation treatment source 102 to position and align radiation treatment source 102 with the target VOI within the patient.

In one embodiment, radiation treatment delivery system 100 may be an image-guided, robotic-based radiation treatment system such as the CyberKnife® system developed by Accuray Incorporated in California. In FIG. 1, radiation treatment source 102 may be a linear accelerator (“LINAC”) mounted on the end of the source positioning system 104 (e.g., robotic arm) having multiple (e.g., 5 or more) degrees of freedom in order to position the LINAC to irradiate a pathological anatomy (target region or volume) with beams delivered from many angles in an operating volume (e.g., a sphere) around the patient. Treatment may involve beam paths with a single isocenter (point of convergence), multiple isocenters, or with a non-isocentric approach (i.e., the beams need only intersect with the pathological target volume and do not necessarily converge on a single point, or isocenter, within the target). Treatment can be delivered in either a single session (non-fraction) or in a small number of sessions (hypo-fractionation) as determined during treatment planning. With radiation treatment delivery system 100, in one embodiment, radiation beams may be delivered according to the treatment plan without fixing the patient to a rigid, external frame to register the intra-operative position of the target volume within the position of the target volume during the pre-operative treatment planning phase.

Imaging sources 108A and 108B and imaging detectors (imagers) 106A and 106B may form an imaging system. In one embodiment, imaging sources 108A and 108B are X-ray sources. In one embodiment, for example, two imaging sources 108A and 108B may be nominally aligned to project x-ray beams through a patient from two differing angular positions (e.g., separated by 90 degrees, 45 degrees, etc.) and aimed through the patient on treatment couch 110 toward respective detectors 106A and 106B. In another embodiment, a single large imager can be used that would be illuminated by each x-ray imaging source. Alternatively, other numbers and configurations of imaging sources and detectors may be used. The imaging detectors 106 are illustrated as being flat (i.e., parallel to the floor and/or flush mounted with the floor), but the imaging detectors 106 may, alternatively, be angled relative to the floor.

A digital processing system may implement algorithms to register images obtained from the imaging system with pre-operative treatment planning in order to align the patient on the treatment couch 110 with the radiation delivery system 100, and to position the radiation treatment source 102 with respect to the target volume precisely. Registration and alignment techniques are known in the art; accordingly, a detailed description is not provided.

In the illustrated embodiment, treatment couch 110 is coupled to a couch position system 112 (e.g., robotic couch arm) having multiple (e.g., 5 or more) degrees of freedom. Couch position system 112 may have five rotational degrees freedom and one substantially vertical, linear degree of freedom. Alternatively, couch positioning system 112 may have six rotational degrees of freedom and one substantially vertical, linear degree of freedom or at least four rotational degrees of freedom. Couch positioning system 112 may be vertically mounted to a column or wall, or horizontally mounted to a pedestal, floor or ceiling. Alternatively, the treatment couch 112 may be a component of another mechanical mechanism, such as a standard treatment couch developed by Accuray Incorporated of California, or be another type of conventional treatment table known to those of ordinary skill in the art.

The treatment delivery system 100 may also include a pad system 602. The pad system 602 is configured to improve patient comfort and/or position a patient, as will be described in further detail hereinafter. The pad system 602 may be separate from or integrated with the treatment couch 112.

A controller 650 is shown operatively coupled to the treatment system 100. The controller 650 may be configured to control the inflation and position of the pad system 602 and/or position of the treatment couch 110 relative to a predefined treatment coordinate system, as described in further detail hereinafter. The controller 650 may also be used to operate the treatment delivery system 100.

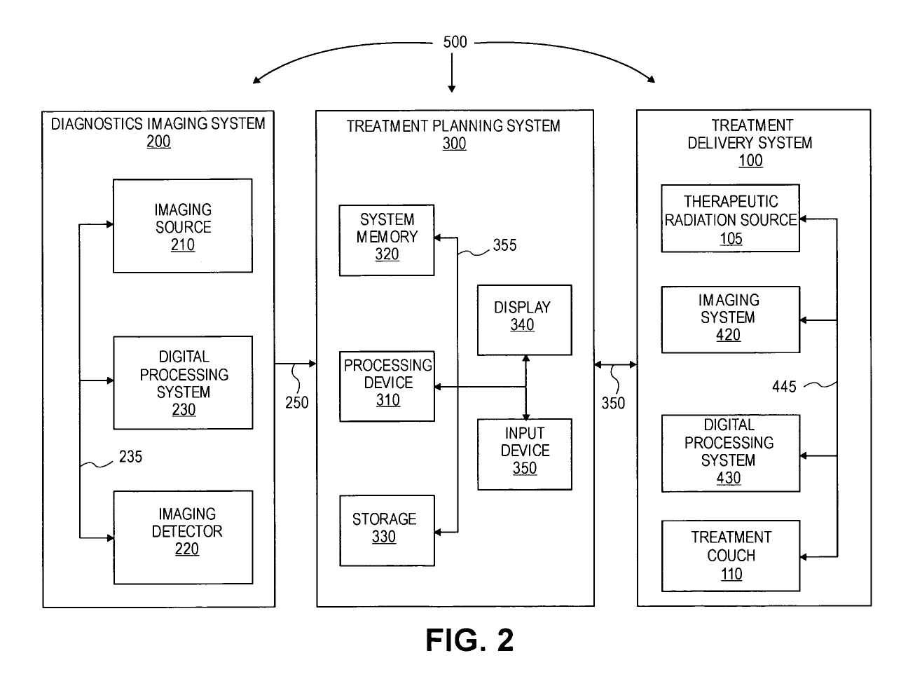

FIG. 2 illustrates one embodiment of systems that may be used to treat a patient in which features of the present invention may be implemented. As described below and illustrated in FIG. 2, system 500 may include a diagnostic imaging system 200, a treatment planning system 300, and a treatment delivery system 100. Diagnostic imaging system 200 may be any system capable of producing medical diagnostic images of a treatment region in a patient that may be used for subsequent medical diagnosis, treatment planning and/or treatment delivery. For example, diagnostic imaging system 200 may be a computed tomography (CT) system, a magnetic resonance imaging (MRI) system, a positron emission tomography (PET) system, an ultrasound system or the like. For ease of discussion, diagnostic imaging system 200 may be discussed below at times in relation to a CT x-ray imaging modality. However, other imaging modalities such as those above may also be used.

Diagnostic imaging system 200 includes an imaging source 210 to generate an imaging beam (e.g., x-rays, ultrasonic waves, radio frequency waves, etc.) and an imaging detector 220 to detect and receive the beam generated by imaging source 210, or a secondary beam or emission stimulated by the beam from the imaging source (e.g., in an MRI or PET scan). In one embodiment, diagnostic imaging system 200 may include two or more diagnostic X-ray sources and two or more corresponding imaging detectors. For example, two x-ray sources may be disposed around a patient to be imaged, fixed at an angular separation from each other (e.g., 90 degrees, 45 degrees, etc.) and aimed through the patient toward (an) imaging detector(s) which may be diametrically opposed to the x-ray sources. A single large imaging detector, or multiple imaging detectors, may also be used that would be illuminated by each x-ray imaging source. Alternatively, other numbers and configurations of imaging sources and imaging detectors may be used.

The imaging source 210 and the imaging detector 220 are coupled to a digital processing system 230 to control the imaging operation and process image data. Diagnostic imaging system 200 includes a bus or other means 235 for transferring data and commands among digital processing system 230, imaging source 210 and imaging detector 220. Digital processing system 230 may include one or more general-purpose processors (e.g., a microprocessor), special purpose processor such as a digital signal processor (DSP) or other type of device such as a controller or field programmable gate array (FPGA). Digital processing system 230 may also include other components (not shown) such as memory, storage devices, network adapters and the like. Digital processing system 230 may be configured to generate digital diagnostic images in a standard format, such as the DICOM (Digital Imaging and Communications in Medicine) format, for example. In other embodiments, digital processing system 230 may generate other standard or non-standard digital image formats. Digital processing system 230 may transmit diagnostic image files (e.g., the aforementioned DICOM formatted files) to treatment planning system 300 over a data link 250, which may be, for example, a direct link, a local area network (LAN) link or a wide area network (WAN) link such as the Internet. In addition, the information transferred between systems may either be pulled or pushed across the communication medium connecting the systems, such as in a remote diagnosis or treatment planning configuration. In remote diagnosis or treatment planning, a user may utilize embodiments of the present invention to diagnose or treatment plan despite the existence of a physical separation between the system user and the patient.

Treatment planning system 300 includes a processing device 310 to receive and process image data. Processing device 310 may represent one or more general-purpose processors (e.g., a microprocessor), special purpose processor such as a digital signal processor (DSP) or other type of device such as a controller or field programmable gate array (FPGA). Processing device 310 may be configured to execute instructions for performing the operations of the treatment planning system 300 discussed herein that, for example, may be loaded in processing device 310 from storage 330 and/or system memory 320.

Treatment planning system 300 may also include system memory 320 that may include a random access memory (RAM), or other dynamic storage devices, coupled to processing device 310 by bus 355, for storing information and instructions to be executed by processing device 310. System memory 320 also may be used for storing temporary variables or other intermediate information during execution of instructions by processing device 310. System memory 320 may also include a read only memory (ROM) and/or other static storage device coupled to bus 355 for storing static information and instructions for processing device 310.

Treatment planning system 300 may also include storage device 330, representing one or more storage devices (e.g., a magnetic disk drive or optical disk drive) coupled to bus 355 for storing information and instructions. Storage device 330 may be used for storing instructions for performing the treatment planning methods discussed herein.

Processing device 310 may also be coupled to a display device 340, such as a cathode ray tube (CRT) or liquid crystal display (LCD), for displaying information (e.g., a two-dimensional or three-dimensional representation of the VOI) to the user. An input device 350, such as a keyboard, may be coupled to processing device 310 for communicating information and/or command selections to processing device 310. One or more other user input devices (e.g., a mouse, a trackball or cursor direction keys) may also be used to communicate directional information, to select commands for processing device 310 and to control cursor movements on display 340.

It will be appreciated that treatment planning system 300 represents only one example of a treatment planning system, which may have many different configurations and architectures, which may include more components or fewer components than treatment planning system 300 and which may be employed with the present invention. For example, some systems often have multiple buses, such as a peripheral bus, a dedicated cache bus, etc. The treatment planning system 300 may also include MIRIT (Medical Image Review and Import Tool) to support DICOM import (so images can be fused and targets delineated on different systems and then imported into the treatment planning system for planning and dose calculations), expanded image fusion capabilities that allow the user to treatment plan and view dose distributions on any one of various imaging modalities (e.g., MRI, CT, PET, etc.). Treatment planning systems are known in the art; accordingly, a more detailed discussion is not provided.

Treatment planning system 300 may share its database (e.g., data stored in storage device 330) with a treatment delivery system, such as treatment delivery system 100, so that it may not be necessary to export from the treatment planning system prior to treatment delivery. Treatment planning system 300 may be linked to treatment delivery system 100 via a data link 350, which may be a direct link, a LAN link or a WAN link as discussed above with respect to data link 250. It should be noted that when data links 250 and 350 are implemented as LAN or WAN connections, any of diagnostic imaging system 200, treatment planning system 300 and/or treatment delivery system 100 may be in decentralized locations such that the systems may be physically remote from each other. Alternatively, any of diagnostic imaging system 200, treatment planning system 300 and/or treatment delivery system 100 may be integrated with each other in one or more systems.

Treatment delivery system 100 includes a therapeutic and/or surgical radiation source 105 to administer a prescribed radiation dose to a target volume in conformance with a treatment plan. Treatment delivery system 100 may also include an imaging system 420 to capture intra-treatment images of a patient volume (including the target volume) for registration or correlation with the diagnostic images described above in order to position the patient with respect to the radiation source. Treatment delivery system 100 may also include a digital processing system 430 to control radiation source 105, imaging system 420, and a patient support device such as a treatment couch 110. Digital processing system 430 may include one or more general-purpose processors (e.g., a microprocessor), special purpose processor such as a digital signal processor (DSP) or other type of device such as a controller or field programmable gate array (FPGA). Digital processing system 430 may also include other components (not shown) such as memory, storage devices, network adapters and the like. Digital processing system 430 may be coupled to radiation source 105, imaging system 420 and treatment couch 110 by a bus 445 or other type of control and communication interface.

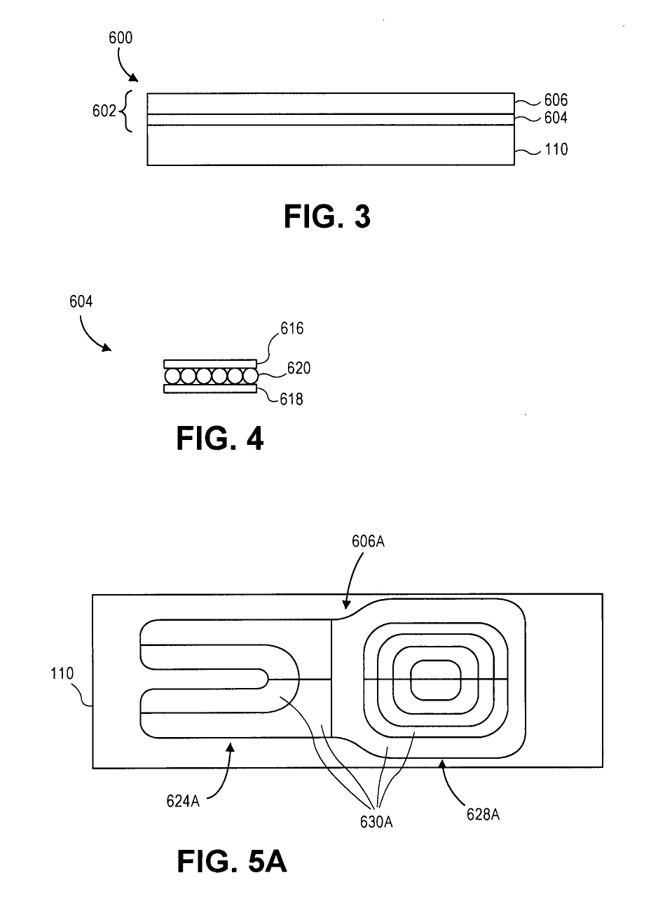

FIG. 3 illustrates a cross-sectional view of a patient positioning system 600 in accordance with one embodiment of the invention. The positioning system 600 includes the treatment couch 110 and a pad system 602. The pad system 602 includes a pressure sensor 604 and a pad 606. In FIG. 3, the pressure sensor 604 and pad 606 are illustrated separately. It will be appreciated that the pressure sensor 604 may be independent of the pad 606 or the pressure sensor may be integrated with the pad 606.

In use, a patient is positioned on the pad 606. The pressure sensor 604 senses the pressure of the patient on the pad 606. In one embodiment, the pressure sensor identifies high stress contact points of the patient. In one embodiment, the pressure sensor may identify that the pressure has exceeded a certain threshold. In another embodiment, the pressure sensor may quantify a specific pressure measurement.

The pad 606 is then inflated based on the pressure distribution of the patient, as will be described in further detail hereinafter. The pad is inflated to improve the comfort of the user. The pad is, therefore, customizable to the patient. The pad may also be inflated to correct patient movement and/or ensure a correct alignment of the patient during treatment. In one embodiment, the pad is inflated with air or other gases. In other embodiments, the pad is inflated with a fluid. The fluid may be a liquid or a gel, such as, for example, water or silicone. It will be appreciated that other materials that allow selective inflation of the pad can be used. The inflation settings of the pad may be stored so the pad can be re-inflated to the stored settings for subsequent treatments.

In one embodiment, the inflation material (e.g., air, other gas, fluid, etc.) is heated. The inflation material may be heated prior to inflation of the pad 606. It will be appreciated that the inflation material may also be heated after inflation of the pad 606. Heating the inflation material may further improve patient comfort. In one embodiment, the inflation material is circulated to maintain heat in the pad 606.

It will be appreciated that the treatment couch 110 and pad system 602 may be separate or integrated. For example, the pad system 602 may be positioned on the treatment couch 110. Alternatively, the pad system 602 may be built-in to the treatment couch 110. In another example, the treatment couch 110 and pad system 602 may be a hybrid system (e.g., the pressure sensor 604 is integrated with the couch 110 and the pad 606 is positioned independently on the treatment couch 110). It will also be appreciated that although the treatment couch 110 is illustrated as a table, the treatment couch 110 and pad system 602 may have different configurations. For example, the treatment couch 110 may be a chair and/or a part of the table may be inclined relative to the floor.

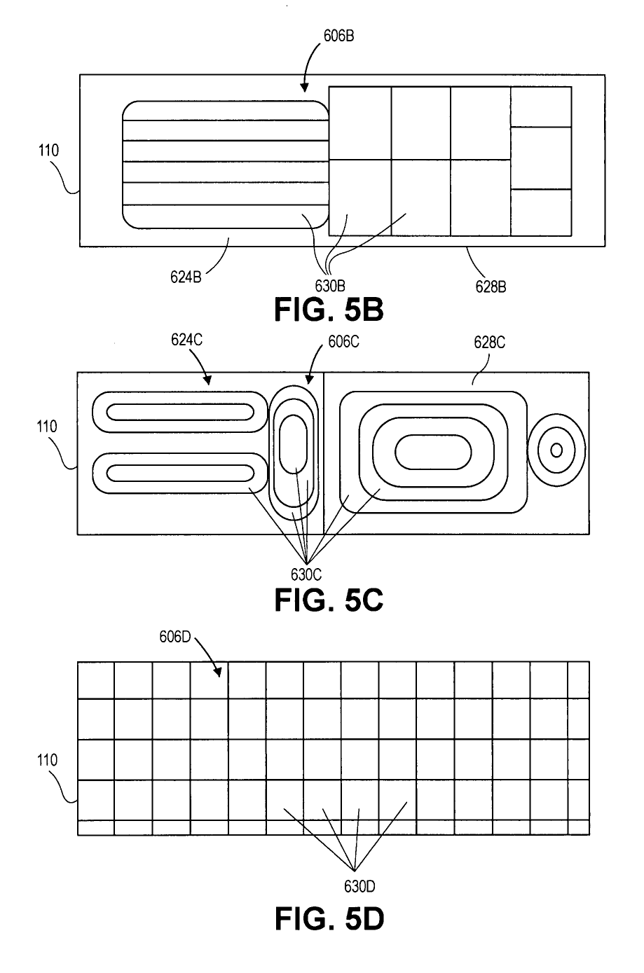

FIG. 4 is a detailed cross-sectional view of the pressure sensor 604. FIG. 4 illustrates an exemplary pressure sensor 604. The pressure sensor 604 includes a first metal layer 616, a second metal layer 618 and an intermediate silicon layer 620 between the metal layers 616 and 618. The pressure sensor 604 measures the displacement of the metal layers 616 and 618 with respect to the intermediate silicon layer 620. It will be appreciated that the pressure sensor may have a different configuration than that illustrated in FIG. 4 and may include different materials than those described above. Other exemplary pressure sensors include strain gauges, capacitive sensors, piezoresistive sensors, and the like.

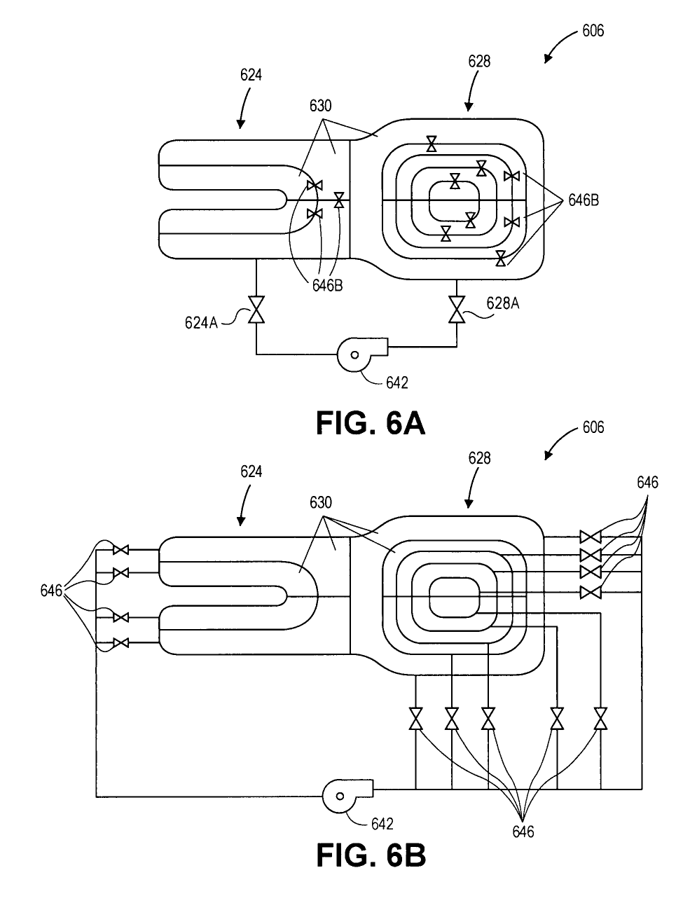

FIGS. 5A-5D illustrate top views of the pad 606 in further detail. The pad 606 is shown positioned on the treatment couch 110. FIGS. 5A-5D are provided to illustrate exemplary configurations of the pad 606; however, it will be appreciated that other configurations may be used for the pad 606 than those illustrated in FIGS. 5A-5D.

In FIG. 5A, the pad 606a is divided into a first zone 624a and a second zone 628a. The first zone 624a corresponds to a lower body portion of a patient and the second zone 628a corresponds to an upper body portion of a patient. The pad 606a includes a plurality of chambers 630a. The chambers 630a are arranged to correspond to the patient's body in the zones 624a, 624b. For example, elongate chambers corresponding to a patient's legs may be provided in the first zone 624a. The zones 624a, 628a may also be separated according to left and right halves of the patient's body. The second zone 628a is shown having several chambers of increasing diameters. The illustrated chambers have a square shape with rounded corners.

In FIG. 5B, the pad 606b is also divided into a first zone 624b and a second zone 628b, corresponding to a lower body portion and an upper body portion of a patient, respectively. The pad 606b includes a plurality of chambers 630a. Each of the chambers in the first zone 624b is the same size and shape. The illustrated chambers in the first zone 624b are elongate and are positioned adjacent one another. Similarly, the chambers in the second zone 628b may have the same size and shape as other chambers in the same zone. In FIG. 5B, an additional chamber is provided corresponding to the patient's head. In FIG. 5B, the chambers are shown having rectangular shapes.

In FIG. 5C, the pad 606c is similarly divided into a first zone 624c and a second zone 628c, corresponding, respectively, to a lower body portion and upper body portion of a patient. The pad 606c includes a plurality of chambers 630c. Each of the zones includes a plurality of chamber regions corresponding to various body parts (e.g., legs, arms, back, head, etc.). Each of the chamber regions is shown having a plurality of chambers of different sizes. The illustrated chambers have a generally elliptical or otherwise rounded shape. In one embodiment, the chamber regions are actively inflated while the chambers within each chamber region are passively inflated, as will be explained hereinafter.

In FIG. 5D, the pad 606d includes a plurality of chambers 630d. Each of the chambers 630d of the pad 606d has the same size and shape throughout the entire pad 606d.

It will be appreciated that the particular configuration of the chambers of the air pad may vary from that illustrated in FIGS. 5A-5D. The number, size and shape of the chambers may also vary from that shown in FIGS. 5A-5D.

The chambers of the air pad can be inflated to improve the comfort of a patient laying on the pad 606. The chambers can also be inflated to improve the stability and/or immobilize the patient. The chambers can also be heated to improve patient comfort. It will also be appreciated that the more comfortable a patient is, the less likely the patient is to move.

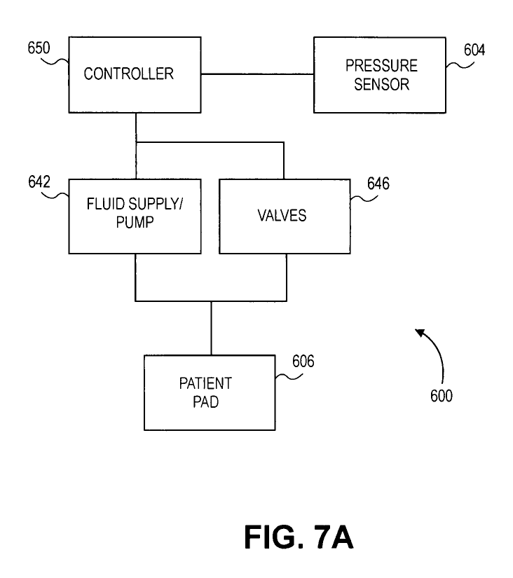

FIGS. 6A-6B illustrate an exemplary mechanical arrangement for the pad 606. The mechanical arrangement is illustrated with the arrangement of pad 606a of FIG. 5A. It will be appreciated that the mechanical arrangements of FIGS. 6A-6B are applicable to pads 606b-d of FIGS. 5B-5D and other configurations of the pad 606.

In FIGS. 6A and 6B, the pad includes a pump 642 in fluid communication with the chambers to selectively inflate the chambers 630 of the pad 606. The pad 606 also includes a plurality of valves 646 in fluid communication with the pump 642 and the chambers 630. In one embodiment, the pump 642 is an air compressor and the valves 646 are solenoid valves. It will be appreciated that other pumps and valves may be used, and that materials other than air may be used, such as, for example, water, gel, other gases, and the like. It will also be appreciated that a fluid reservoir may also be provided in fluid communication with the pump 642, valves 646 and chambers 630.

In FIG. 6A, two primary valves 646a are provided in fluid communication with each zone 624, 626. Within each zone 624, 626, secondary valves 646b are provided to fluidly connect the chambers within the zone. Thus, each zone 624, 626 is in direct communication with the pump 642, while the chambers within the zone are indirectly connected to the pump 642. In FIG. 6B, each chamber is in direct fluid communication with the pump 642. A valve 646 is provided between each of the chambers 630 and the pump 642. It will be appreciated that the configuration of the valves and pump may vary from that illustrated in FIGS. 6A-6B. For example, more than one pump may be provided, one or more valves may be provided for each chamber, and the like.

In either configuration, each of the valves can be actively controlled to fill its associated chamber. Alternatively, some of the valves may be actively controlled to fill their associated chambers, while others are not controlled. Thus, the chambers that are associated with an actively controlled valve are actively inflated, while the remaining chambers are passively inflated. For example, in the embodiment of FIG. 5A, the primary valves 646a can be actively controlled while the secondary valves 646b are passively controlled.

It will be appreciated that in embodiments in which the inflation material is heated, a heat transfer system (not shown) may also be operatively coupled to the pad 606 in a variety of ways. Systems for heating air and fluids are well-known in the art; accordingly, a more detailed discussion is not provided. It will be appreciated that the pump 642 can be activated to circulate the heated inflation material to maintain heat in the pad 606.

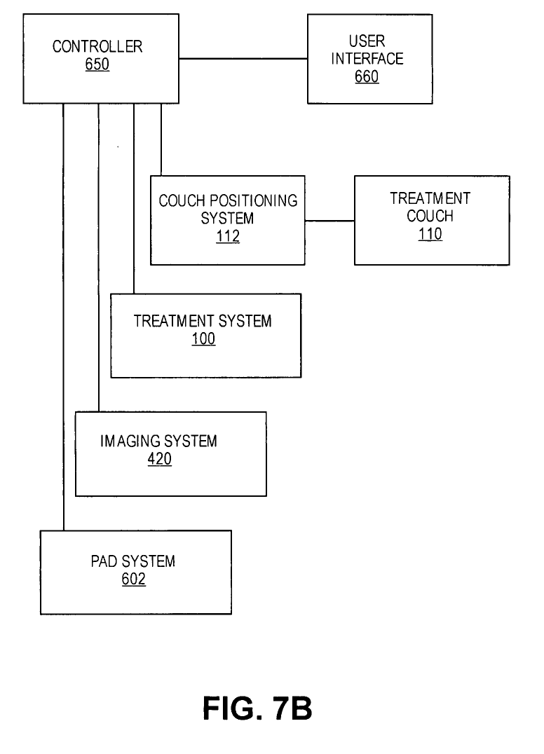

FIG. 7A is a block diagram of a patient positioning system 600 in accordance with one embodiment of the invention. The patient positioning system 600 includes a controller 650. The controller 650 receives pressure data from the pressure sensor 604. The controller 650 analyzes the pressure data and determines inflation settings for the pad 606. The controller 650 activates the pump 642 and valves 646 to control the inflation of the chambers of the pad 606 according to the inflation settings. In one embodiment, the controller 650 controls heating of the inflation material.

FIG. 7B is a block diagram of a patient positioning system 600 in accordance with one embodiment of the invention. In FIG. 7B, the controller 650 is operatively coupled to pad system 602, the couch positioning system 112 and treatment couch 110, the treatment system 100 and the imaging system 420. The controller 650 may also be operatively coupled to a user interface 660, as described in further detail hereinafter. In one embodiment, the controller 650 calculates the position of the pad 602 relative to the treatment room or other predefined treatment coordinate system. The controller 650 may also operate to control the motion of the treatment system 100 and/or couch positioning system 112 in a way that a treatment target within the patient's anatomy remains properly aligned with respect to a treatment beam source of the treatment system 100 throughout the treatment procedure. Similarly, the controller 650 may operate to control the inflation of the pad 606 to maintain proper alignment. The controller 650 may also be used to operate the treatment system 100. The controller 650 may also communicate with the treatment system 100, receiving pre-treatment scan data representative of one or more pre-treatment scans of a treatment target within the patient. The pre-treatment scans may show the position and orientation of the target with respect to a pre-treatment coordinate system. The controller 650 may also receive from the imaging system 420 image data representative of real time or near real time images of the target. The image data may contain information regarding the real time or near real time position and orientation of the target with respect to a treatment coordinate system. The treatment coordinate system and the pre-treatment coordinate system are related by known transformation parameters.

The controller 650 may include an input module for receiving 1) pre-treatment scan data representative of pre-treatment scans of the target, and 2) real time or near real time image data representative of real time or near real time images of the target. The pre-treatment scans show the position and orientation of the target with respect to the pre-treatment coordinate system. The near real-time images, taken by the imaging system 420 under the command of the controller 650, show the position and orientation of the treatment target with respect to the treatment coordinate system. The treatment coordinate system and the pre-treatment coordinate systems are related by known transformation parameters. The controller 650 includes a TLS (target location system) processing unit that computes the position and orientation of the treatment target in the treatment coordinate system, using the pre-treatment scan data, the real time or near real time image data, and the transformation parameters between the pre-treatment coordinate system and the treatment coordinate system. The processing unit of the controller 650 may also compute the position and orientation of the iso-center of the treatment system 100.

The treatment system 100 may include a sensor system for detecting the position of the treatment couch 110 and/or pad system 602. The sensor system may be a resolver-based sensor system. Alternatively, other sensor systems known by those skilled in the art may be used, such as an inertial sensor attached to the treatment couch 110 and/or pad system 620 for sensing the motions of the treatment couch 110 and/or pad system 602, or an infrared triangulation system, or a laser scanning system or an optical tracking system disposed within the treatment room for detecting the position of the treatment couch 110 and/or pad system 602 relative to the treatment room or other treatment coordinate system, or an optical encoder.

An exemplary laser scanning system may scan the treatment room approximately 60x/sec to determine the position of the treatment couch 110 and/or pad system 602. The laser scanning system may include devices performing a single plane scanning, or two-plane scanning, or multiple-plane scanning. Correspondingly, the controller 650 may be loaded with software adapted for receiving information from the sensor system and calculating the position of the patient treatment couch 110, pad system 602, as well as the treatment system 100, so that the controller 650 always knows the position of the treatment couch 110 and/or pad system 602. The controller 650 may be programmed to automatically or periodically calibrate the treatment couch 110 and/or pad system 602 with the therapeutic radiation source of the treatment system 100. In an alternate embodiment, the sensor system includes a magnetic tracking system for tracking the position of the treatment couch 110 and/or pad system 602 relative to the treatment coordinate system. The magnetic tracking system preferably includes at least one transducer attached to the treatment couch 110 and/or pad system 602.

The controller 650 may be adapted to detect a misalignment of the treatment target with the iso-center of the radiation source caused by a patient's movement by comparing the position of the treatment target with the iso-center of the radiation source, and generate motion command signals for implementing corrective motions of the treatment system 100 and/or couch positioning system 112 and/or implementing corrective volume changes of the chambers of the pad 606 for aligning the treatment target with respect to the radiation treatment source of the treatment system 100.

In another embodiment, the corrective motions of one or more of the treatment system 100, couch positioning system 112 and pad system 602 may accommodate for various motions, such as respiratory motion; cardiac pumping motion of the patient's heart; sneezing, coughing, or hiccupping; and muscular shifting of one or more anatomical members of the patient.

In another embodiment, one or more of the treatment system 100, couch positioning system 112 and pad system 602 including the controller 650 may be adapted to detect and accommodate changes in tumor geometry that may be caused by tissue deformation by comparing the real time or near real time image with the pre-treatment image and repositioning the patient using the pad system 602 and/or treatment couch 110 and/or the radiation source of the treatment system 100 (in a robot-based therapeutic radiation treatment system), or adjusting the volume of the chambers of the pad system 602, positions of the treatment couch 110 and the radiation source of the treatment system 100 to correspond to the treatment plan.

The controller 650 includes software for establishing and maintaining a reliable communication interface with the pad system 602, couch positioning system 112 and treatment system 100. In one embodiment, the software uses the interface specifications developed for the pad system 602. The controller 650 further includes software for converting the patient position and orientation information from the imaging system 420 to appropriate units of volume for the pad system 602 and/or appropriate units of movement in the degrees of freedom of motion capability of the treatment couch 110. The controller 650 may include software for providing a user interface unit 660 to the treatment system user control console, to monitor and initiate the motion of the pad system 602 for positioning the patient. The controller 650 may also include software for detecting, reporting, and handling errors in communication or software control of the pad system 602.

The controller 650 may include at least one user interface unit, such as user interface unit 660, for enabling the user to interactively control the motions or corrective motions of the pad system 602, by implementing one or more user-selectable functions. In one embodiment, the user interface unit 660 may be a handheld user interface unit or remote control unit. Alternatively, the user interface unit 660 may be a graphical user interface (GUI).

The communication links between the controller 650 and other components of the patient positioning system 600 may be wired links or wireless links, with a bandwidth necessary for maintaining reliable and timely communications.

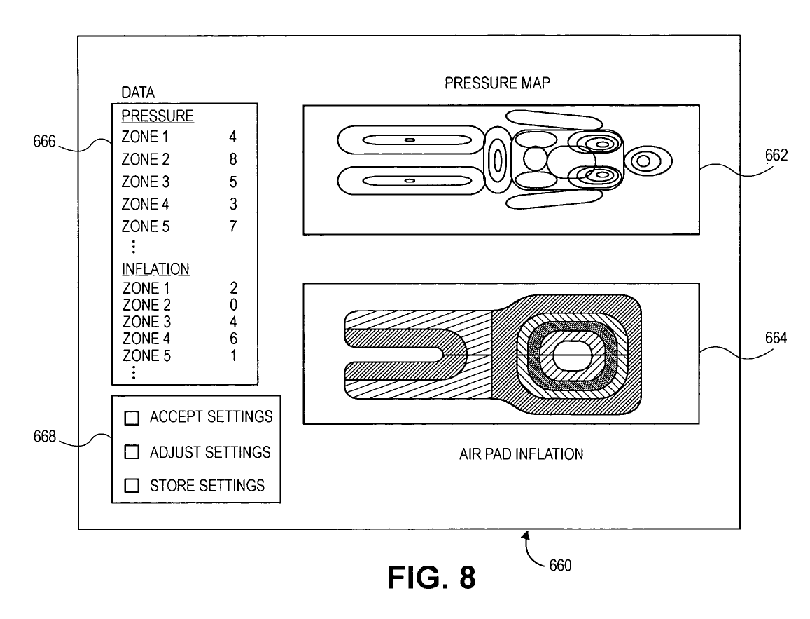

FIG. 8 is an exemplary screen shot of a user interface 660 for use with the patient positioning system 600. It will be appreciated that the user interface and screen shots may vary from those illustrated and described. As shown in FIG. 8, the interface 660 may include a pressure map 662 of the patient using data from the pressure sensor 604. The interface 660 may also include recommended inflation settings 664 for the pad 606. The interface 660 may also include specific data 666 about the pressure map 662 and/or inflation settings 664. The interface 660 may include an interactive box 668 in which a user may choose to select the settings as determined by the system, adjust the settings determined by the system, and/or store the settings. In one embodiment, the user interface 660 is incorporated into the treatment planning system 300 and/or treatment delivery system 100 of FIG. 2.

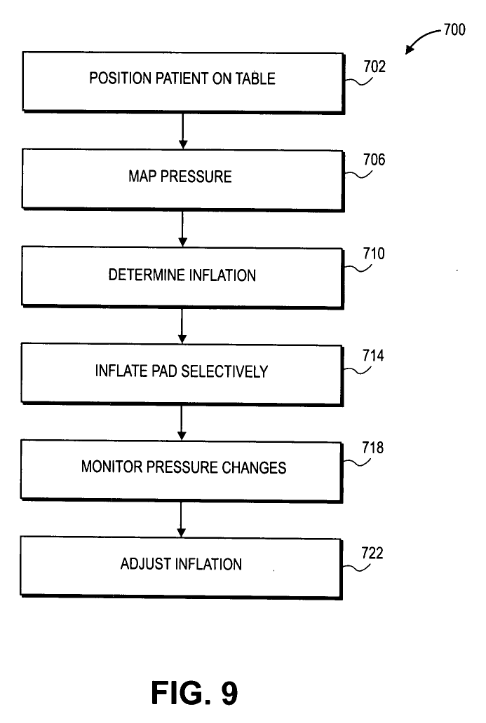

FIG. 9 shows a process 700 for positioning a patient in accordance with one embodiment of the invention. The process begins at block 702 by positioning a patient on the pad system 602. As discussed above, the patient may be positioned on the pad system 602 during a treatment planning and/or delivery process. It will be appreciated that the patient may be positioned on the pad system 602 during other aspects of a treatment process including, for example, imaging or treatment delivery.

The process continues at block 706 by mapping the pressure of the patient on the pad system 602. The pressure is measured by the pressure sensor 604.

The process continues at block 710 by determining inflation settings for the pad 606. As discussed above, the controller 650 analyzes the pressure points as measured by the pressure sensor and determines the inflation settings that will provide comfort for the patient (e.g., by minimizing pressure points). The inflation settings can also be stored. In one embodiment, the inflation settings are stored for subsequent treatment steps. The inflation settings can also be manually adjusted. For example, if a patient is uncomfortable with the inflation settings, the inflation settings can be adjusted manually until the patient is comfortable.

The process continues at block 714 by selectively inflating the chambers of the pad 606 in accordance with the inflation settings. The pump 642 and valves 646 are activated to inflate the pad 606 as required by the inflation settings. In one embodiment, the inflation material is heated and/or circulated throughout the pad 606 during planning and/or treatment.

In one embodiment, the process continues by monitoring pressure changes at block 718. The process may continue by adjusting the inflation of the chambers at block 722. The pressure sensor can be used to monitor changes in pressure. For example, if a patient moves during treatment, the pressure sensor senses the changes and the controller can selectively inflate chambers to return the patient to their original position. If a chamber on a right side of a patient is inflated and/or a chamber on the left side of the patient is deflated, then the patient will be rolled counter-clockwise if the pressure sensor 604 detects that the patient has rolled clockwise. The adjustment may be automatic and dynamic. That is, the controller may automatically adjust the inflation of the chambers in real-time in response to pressure changes detected by the pressure sensor 604.

It should be noted that the methods and apparatus described herein are not limited to use only with radiation treatment systems. It will be appreciated that the methods and apparatus can be used in other surgical systems, medical imaging systems, or any other system in which a patient is required to sit in a chair or lie down for extended periods of time, such as, for example, a CT x-ray machine, MRI machine, etc. In alternative embodiments, the methods and apparatus herein may be used in applications outside of the medical field, such as, for example, in the automotive industry. For example, car seats may include the pad system 602 or the pad system 602 can be positioned on a car seat to improve the comfort of a driver and/or passengers of a car or truck.

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

What is claimed is:

1. A system comprising:
a pressure sensor to sense a relative pressure of a body on the pressure sensor; and
a pad having a plurality of selectively inflatable chambers disposed on the pressure sensor.

2. The system of claim 1, wherein the pressure sensor and the pad are positioned on a table of a radiation treatment system.

3. The system of claim 1, further comprising a table, wherein the pressure sensor and the pad are positioned on the table.

4. The system of claim 1, further comprising a table, wherein the pressure sensor and the pad are integrated with the table.

5. The system of claim 1, further comprising a controller coupled to the pressure sensor to monitor pressure measured by the pressure sensor and selectively inflate each chamber of the pad.

6. The system of claim 5, wherein the controller is further configured to monitor changes in pressure measured by the pressure sensor.

7. The system of claim 6, wherein the controller is configured to selectively adjust the inflation of each chamber.

8. The system of claim 1, further comprising a pump in fluid communication with the pad.

9. The system of claim 1, further comprising a plurality of valves, each of the plurality of chambers fluidly connected to least one of the plurality of valves.

10. The system of claim 1, wherein the pad comprises a first zone corresponding to an upper body portion of the patient and a second zone corresponding to a lower body portion of the patient.

11. The system of claim 10, wherein the first zone comprises a first plurality of bladders and the second zone comprises a second plurality of bladders.

12. The system of claim 10, wherein the first zone is fluidly separated from the second zone.

13. The system of claim 1, wherein the pad is inflated with air.

14. The system of claim 1, wherein the pad is inflated with a fluid.

15. The system of claim 5, wherein the controller is configured to create a pressure map of the sensed pressure.

16. The system of claim 1, wherein the pad is inflated with a heated inflation material.

17. The system of claim 16, wherein the heated inflation material is circulated through the pad.

18. A patient positioning pad comprising:
a plurality of chambers configured to be positioned on a table, each of the plurality of chambers selectively inflatable.

19. The pad of claim 18, further comprising a pressure sensor.

20. The pad of claim 18, further comprising a plurality of pressure sensors, each of the plurality of chambers comprising at least one of the plurality of pressure sensors.

21. The pad of claim 18, wherein the plurality of chambers are inflated with air.

22. The pad of claim 18, wherein the plurality of chambers are fluidly separated into a first zone and a second zone, the first zone corresponding to an upper body portion of a patient and the second zone corresponding to a lower body portion of a patient.

23. The pad of claim 18, wherein the inflation of each of the plurality of chambers is adjustable.

24. The pad of claim 23, wherein the inflation of each of the plurality of chambers is automatically adjustable.

25. The pad of claim 23, wherein the inflation of each of the plurality of chambers is automatically adjustable.

26. The pad of claim 18, wherein each of the plurality of chambers is inflatable with a heated inflation material.

27. A method comprising:
sensing a relative pressure of a body; and
selectively inflating chambers of a pad having a plurality of chambers in response to the sensed relative pressure.

28. The method of claim 27, further comprising sensing changes in pressure of the body and adjusting the inflation of the plurality of chambers in response to the sensed changes in pressure.

29. The method of claim 27, wherein the pressure is sensed at a plurality of locations and chambers corresponding to the plurality of locations are selectively inflated in response to the sensed pressure.

30. The method of claim 27, further comprising storing data corresponding to the selective inflation of the plurality of chambers.

31. The method of claim 30, further comprising inflating the plurality of chambers using the stored data.

32. The method of claim 27, further comprising creating a pressure map of the sensed relative pressure.

33. The method of claim 27, further comprising:
determining a position and orientation of a treatment target with respect to a pre-treatment coordinate system;
determining a near real time position and orientation of the treatment target with respect to a treatment coordinate system, the treatment coordinate system having a predetermined relationship to the pre-treatment coordinate system; and
determining one or more inflation adjustments of the chambers of the pad to substantially match the position and orientation of the treatment target in the pre-treatment coordinate system of the target with the position and orientation of the treatment target in the treatment coordinate system.

34. The method of claim 33, further comprising determining one or more corrective motions of a support device to move the support device with respect to the therapeutic radiation source and determining one or more inflation adjustments of the chambers of the pad to substantially match the position and orientation of the treatment target in the pre-treatment coordinate system of the target with the position and orientation of the treatment target in the treatment coordinate system.

35. The method of claim -34, wherein the pad is positioned on the support device.

36. The method of claim 27, wherein the chambers are selectively inflated with a heated inflation material.

37. The method of claim 37, further comprising circulating the heated inflation material through the pad.

38. A system comprising:
means for sensing a relative pressure of a body; and
means for selectively inflating chambers of a pad having a plurality of chambers in response to the sensed relative pressure.

39. The system of claim 38, further comprising means for storing data corresponding to the selective inflation of the plurality of chambers.

40. The system of claim 38, wherein the means for sensing a relative pressure of a body comprises means for sensing changes in pressure of the body and adjusting the inflation of the plurality of chambers in response to the sensed changes in pressure.

41. The system of claim 38, further comprising means for creating a pressure map of the sensed relative pressure.
